(19) United States
(12) Patent Application Publication
(10) Pub. No.: US 2003/0026819 A1
(43) Pub. Date: Feb. 6, 2003

(54) Cream-to-powder dermatological composition

(76) Inventors: Luis A. Rios, Penbroke Pines, FL (US); Ramon Figueroa, Hollywood, FL (US)

Correspondence Address: JOHN S. PRATT, ESQ, KILPATRICK STOCKTON, LLP, 1100 PEACHTREE STREET, SUITE 2800, ATLANTA, GA, 30309, US

(21) Appl. No.: 10/096,085

(22) Filed: Mar. 11, 2002

Related U.S. Application Data
(60) Provisional application No. 60/274,515, filed on Mar. 9, 2001.

(51) Int. Cl. A61K 7/00

(52) U.S. Cl. 424/401

(57) ABSTRACT

A cream, comprising about 5 to 60% of a non-swelling clay, a swelling clay, a natural or synthetic gum, a surfactant, a hydrophilic and/or hydrophobic solvent, wherein, after application to a surface, dries to a uniform, visible, substantive, abrasion-resistant powder. The cream/powder composition is useful for a protective skin covering for preventing conditions such as bed sores, diaper rash, poison ivy or soothing uncomfortable feet, or as a delivery vehicle for a therapeutically-active dermatological agent, such as acne or fungal treatments, or as the delivery vehicle for treating other types of conditions, such a sore muscles, sore joints or microbial infections. Methods of making and using the cream also are included.

### TECHNICAL AREA

The present invention relates to a cream-to-powder dermatological composition that during initial application to the skin spreads as a conventional cream, gel, or ointment, and shortly after application dries to a uniform, visible, substantive abrasion-resistant powder. The cream-to-powder dermatological composition may be used as protection of skin against such disorders as bed sores, contact dermatitis, poison ivy and diaper rash. A therapeutically-active agent may also be added, wherein the cream/powder composition provides stationary dosing of an active agent.

### BACKGROUND

A dermatological composition, with or without a therapeutically active agent, may be topically applied to the skin in a variety of formulations including a liquid, lotion, suspension, gel, balm, salve, ointment, emulsion, stick, spray, cream and powder. Cream formulations, because of their ease of application, are preferred topical formulations. However, cream formulations present problems including, but not limited to, greasiness, removal by contact, staining of clothing, evaporating from the skin, vanishing on the skin and drying to a scaly crust. Those cream formulations that evaporate from and vanish on the skin preclude visual monitoring of the maintenance of the applied cream formulation on the skin, and furthermore preclude from maintaining an active agent in a stationary position on the skin.

Powder formulations, because they are not greasy, do not stain clothing, temporarily do not evaporate from the skin or vanish from the skin and do not dry to a scaly crust, also are acceptable topical formulations. However, powder formulations present problems including, but not limited to, difficulty of application to a defined area, dispersion of volatile particles into the atmosphere and loss of the therapeutic activity on the skin, particularly if the case where therapeutic active is in a vehicle carrier are in an insoluble suspension. Therefore powder formulations preclude long-term visual monitoring of the maintenance of the applied powder formulation on the skin, and furthermore preclude from maintaining an active agent in a stationary position on the skin. For example, a commercial product (Dr. Scholl™) containing a miconazole nitrate in a spray powder is not substantive enough to deliver and keep the active agent in a stationary position as the powder is not sufficiently substantive and abrasive resistant.

Suspensions in general, be they in the form of a cream, gel, ointment, or powder, that act as carriers of insoluble active agents, tend to evaporate or migrate from the original application site, leaving insoluble active agents in a non-stationary position on the skin. Therefore, there is a need for a topical therapeutic composition that can be applied to a visually defined area and that remains visually defined for long periods of time on the skin. Furthermore there is a need for the same therapeutic composition to deliver therapeutically active agents in a stationary manner, particularly when deliver insoluble active agents. The topical therapeutic composition would ideally combine the ease of application of a cream, gel, or ointment with non-greasy, non-staining, non-evaporating, non-vanishing, non-volatile, and non-scaling properties.

### SUMMARY OF THE INVENTION

This invention fulfills these needs by providing a hydrophilic, easy to apply spread-able cream composition (hereinafter, “cream”) comprising about 5 to 60% of a non-swelling clay, a swelling clay, a natural or synthetic gum, and a natural of synthetic film former, such that, after application the cream dries to a uniform, visible, substantive, abrasion-resistant dry powder layer (hereinafter, “visible powder layer”). Optional ingredients also include a non-ionic, or anionic or cationic surfactant, a hydrophilic or hydrophobic solvent, and a therapeutically active agent (hereafter, “cream-active agent”). Methods of making the cream and the cream-active agent are included.

The cream-active agent of the present invention is particularly useful for dermatological applications and in particular for the treatment of dermatological and other disorders, and as a protective agent for the skin. The cream-active agent of the present invention, when applied to a defined area of a surface including, but not limited to, the skin, dries to a defined visible powder layer that remains on the surface until removed by a mild-surfactant and water, or by a strong surfactant and water, or by a mild dermatological cleansing lotion such as Cetaphyl Cleansing Lotion™ (Beiersdorf). The visible powder layer is non-greasy, non-staining, non-evaporating, non-vanishing, particulate non-volatile, non-scaling and enables visual identification and monitoring of the defined area to which the cream-active agent was applied. Furthermore, any active agent that is delivered by the cream-active agent is delivered in a stationary location within the skin surface. Moreover, the visible powder layer formed by the cream-active agent provides a vehicle from which an effective amount of the therapeutically active agent is delivered in a stationary location for immediate and/or during an extended time onto or into a surface.

Accordingly, it is the object of the present invention is to provide an easy to apply spread-able cream-active agent for application on the skin of an animal, including a human, that dries to a visible powder layer.

Another object of the present invention is to provide an easy to apply spread-able cream-active agent for application on a surface that that dries to a visible powder layer and that remains visible on the surface.

Another object of the present invention is to provide an easy to apply spread-able cream-active agent for application on the skin of an animal, including a human, that dries to a visible powder layer and enables visual identification of the area of application for the time it remains on the skin.

Another object of the present invention is to provide an easy to apply spread-able cream-active agent for application on a surface that dries to a visible powder layer and that remains visible on the surface until removed with a mild or strong surfactant or by a mild dermatological cleansing lotion such as Cetaphyl Cleansing Lotion™ (Beiersdorf).

Another object of the present invention is to provide an easy to apply spread-able cream-active agent for application on a surface that dries to a visible powder layer and that remains visible on the surface until removed with a strong surfactant, for use with uncooperative patients, such as children, pets, livestock or difficult adult patients.

Another object of the present invention is to provide an easy to apply spread-able cream-active agent for application on the skin of an animal, including a human, that dries to a visible powder layer that acts as a carrier for delivering stable therapeutically active agents.

Another object of the present invention is to provide an easy to apply spread-able cream-active agent for application on the skin of an animal, including a human, that dries to a visible powder layer that can act as a carrier of anionic, cationic, amphoteric, and non-ionic therapeutically active agents.

Another object of the present invention is to provide an easy to apply spread-able cream-active agent for application as a therapeutic treatment on the skin of an animal, including a human, that dries to a visible powder layer and that enables visual monitoring of therapeutic time of action on the area of application for the time it remains on the skin.

Another object of the present invention to invention to provide an easy to apply spread-able cream-active agent for application as a therapeutic treatment on the skin of an animal, including a human, that dries to a visible powder layer and will not cause irritation on the skin for the time it remains on the skin.

Another object of the present invention is to provide an easy to apply spread-able cream-active agent for application on the skin of an animal, including a human, that dries to a visible powder layer that can act as a carrier for delivering a therapeutically active agents to a stationary location of the skin.

Another object of the present invention is to provide an easy to apply spread-able cream-active agent for application on the skin of an animal, including a human, that dries to a visible powder layer and that enables treatment of skin related disorders.

Another object of the present invention is to provide an easy to apply spread-able cream-active agent for application on the skin of an animal, including a human, that dries to a visible powder layer and that is applied to small, defined areas of the skin.

Another object of the present invention is to provide an easy to apply spread-able cream-active agent for application on the skin of an animal, including a human, that dries to a visible powder layer and that is applied to large defined areas of the skin.

Another object of the present invention to invention is to provide an easy to apply spread-able cream-active agent for application on a surface that dries to a visible powder layer and that delivers therapeutically active agents onto the skin.

Another object of the present invention is to provide an easy to apply spread-able cream-active agent for application on the skin of an animal, including a human that dries to a visible powder layer and that delivers the therapeutically active agents into the skin.

Another object of the present invention is to provide an easy to apply spread-able cream-active agent for application on the skin of an animal, including a human, that dries to a visible powder layer and that enables simple, safe and non-invasive administration of therapeutically active agents onto the skin.

Another object of the present invention is to provide an easy to apply spread-able cream-active agent for application on the skin of an animal, including a human, that dries to a visible powder layer and that enables simple, safe and non-invasive administration of therapeutically active agents into skin.

Another object of the present invention is to provide an easy to apply spread-able cream-active agent for application on the skin of an animal, including a human, that dries to a visible powder layer and enables delivery of therapeutically active agents onto the skin immediately.

Another object of the present invention is to provide an easy to apply spread-able cream-active agent for application on the skin of an animal, including a human, that dries to a visible powder layer and enables delivery of therapeutically active agents into the skin immediately.

Another object of the present invention is to provide an easy to apply spread-able cream-active agent for application on the skin of an animal, including a human, that dries to a visible powder layer and that enables delivery of therapeutically active agent onto the skin during an extended time.

Another object of the present invention is to provide an easy to apply spread-able cream-active agent for application on the skin of an animal, including a human, that dries to a visible powder layer and that enables delivery of therapeutically active agents into the skin during an extended time.

Another object of the present invention is to provide an easy to apply spread-able cream-active agent for application on the skin of an animal, including a human, that dries to a visible powder layer and that has skin-protection properties.

Another object of the present invention to invention to provide an easy to apply spread-able cream-active agent for application on the skin of an animal, including a human, that dries to a visible powder layer and that promotes wound healing.

Another object of the present invention to invention to provide an easy to apply spread-able cream-active agent for application on the skin of an animal, including a human, that dries to a visible powder layer and that promotes relief of inflammatory skin conditions.

Another object of the present invention to invention to provide an easy to apply spread-able cream-active agent for application on the skin of an animal, including a human, that dries to a visible powder layer and promotes analgesic effects.

Another object of the present invention is to provide an easy to apply spread-able cream-active agent for application on the skin of an animal, including a human, that dries to a visible powder layer and that enables simple, safe administration of therapeutically active agents into skin to deliver vasodialator agents to increase microcirculation to prevent or relieve incidents of dibicutis ulcers from forming in bed-ridden patients.

Another object of the present invention to invention to provide an easy to apply spread-able cream-active agent for application on the skin of an animal, including a human, that dries to a visible powder layer and promotes relief of allergic itchy skin conditions.

Another object of the present invention to invention to provide an easy to apply spread-able cream-active agent for application on the skin of an animal, including a human, that dries to a visible powder layer and promotes anti-microbial effects.

Another object of the present invention to invention to provide an easy to apply spread-able cream-active agent for application on the skin of an animal, including a human, that dries to a visible powder layer and promotes keratolytic effects.

Another object of the present invention to invention to provide an easy to apply spread-able cream-active agent for application on the skin of an animal, including a human, that dries to a visible powder layer and promotes skin lightning effects for hyper-pigmentation disorders.

Another object of the present invention to invention to provide an easy to apply spread-able cream-active agent for application on the skin of an animal, including a human, that dries to a visible powder layer and promote relief for excessive sebaceous gland secretion.

Another object of the present invention to invention to provide an easy to apply spread-able cream-active agent for application on the skin of an animal, including a human, that dries to a visible powder layer and promotes protection against fecal microorganisms.

Another object of the present invention to invention to provide an easy to apply spread-able cream-active agent for application on the skin of an animal, including a human, that dries to a visible powder layer and that acts as skin protective or prophylactic layer against a covering.

Another object of the present invention to invention to provide an easy to apply spread-able cream-active for application on the skin of the hands that dries to a visible powder layer and that acts as a skin protective or prophylactic layer against induced contact dermatitis.

Another object of the present invention to invention to provide an easy to apply spread-able cream-active agent for application on the skin of the hands that dries to a visible powder layer and that acts as a skin protective or prophylactic layer against gloves.

Another object of the present invention to invention to provide an easy to apply spread-able cream-active for application on the skin of the hands that dries to a visible powder layer and that acts as a skin protective or prophylactic layer against glove induced contact dermatitis.

Another object of the present invention to invention to provide an easy to apply spread-able cream-active agent for application on the skin of the hands that dries to a visible powder layer and that acts as a visual indicator of glove perforation or breakage.

Another object of the present invention to invention to provide an easy to apply spread-able cream-active agent for application on the skin of the hands that dries to a visible powder layer and that provides protection against microbial contamination resulting from glove perforation or breakage.

Another object of the present invention to invention to provide an easy to apply spread-able cream-active agent for application on the skin that dries to a visible powder layer and that provides protection against microbial contamination.

Another object of the present invention to invention to provide an easy to apply spread-able cream-active agent for application on the skin of the hands that dries to a visible powder layer and that provides protection against microbial contamination and provides visual warning against blood contamination resulting from glove perforation or breakage.

Another object of the present invention to invention to provide an easy to apply spread-able cream-active agent for application on the skin of the hands that dries to a visible powder layer and that provides microbial contamination and provides visual warning against contamination resulting from glove perforation or breakage by incorporating color indicators into the cream active agent.

These and other objects, features and advantages of the present invention will become apparent after a review of the following detailed description of the disclosed embodiments and the appended claims.

### DETAILED DESCRIPTION OF THE INVENTION

The present invention provides a cream comprising about 5 to 60% of a non-swelling clay, a swelling clay, a natural gum, a non-ionic surfactant and a hydrophilic and/or hydrophobic solvent, wherein, after application to a surface, the cream dries to a visible powder layer. Further, the present invention provides a cream-active agent comprising about 5 to 60% of a non-swelling clay, a swelling clay, a natural or synthetic gum, a non-ionic surfactant, a natural or synthetic film former, a hydrophilic solvent, a hydrophobic solvent, or combinations thereof, and a therapeutically active agent, wherein, after application to a surface, the cream dries to visible powder layer from which an effective amount of the active agent is delivered onto or into the surface immediately and/or over an extended time.

As used herein, the term “active agent” includes any natural or synthetic agent, any pharmaceutical agent that treats the disorder for which it is applied.

As used herein, the term “pharmaceutical” includes any natural or synthetic agent approved by a regulatory agency of a country or a state government for the treatment of a disorder or listed in the U.S. Pharmacopoeia (USP) or other generally recognized pharmacopoeia for use in an animal, including a human.

As used herein, the term “stable therapeutically-active agents” includes any natural or synthetic agent approved by a regulatory agency of a country or a state government or listed in the U.S. Pharmacopoeia (USP) or other generally recognized pharmacopoeia for use in an animal, including a human, that when used in a composition, is stable under pharmaceutically accepted standards of accelerated storage conditions.

As used herein, the word “substantive” is defined as a property of the dried phase of the dermatological cream of the present invention, the powder, wherein the powder can not be rubbed off by daily abrasive conditions, such as rubbing against clothing, wearing shoes and socks all day, or attempting to rub off with the hands. The cream-active agent of the present invention is substantive, wherein the cream-active agent is not easily removable unless when removed with at least one removing agent, such as with a surfactant and water, preferably a mild surfactant and water. It is to be understood that while a small amount of the composition of the invention may be rubbed or scratched off, a majority of the composition will remain at the location of application until it is washed off with water and the removing agent.

As used herein, the word “skin” includes the epidermis, dermis, sebaceous glands and mucus membranes of an animal, including a human. Mucous membranes include, but are not limited to, vaginal mucosa to promote moisture absorption and reduce high moisture environment that is conducive to yeast viability.

As used herein, the term “hydrophobic solvent” includes any solvent that repels water molecules.

As used herein, the term “hydrophilic solvent” includes any solvent that repels lipid molecules.

As used herein, the term “extended time” refers to a period of time that allows at least a twofold reduction in dosing frequency of an active agent as compared to the active agent presented in a conventional dosage form. (United States Pharmcopeia- USP XXI, Preface, pg. XLIII).

In contrast to prior art methods, wherein either a cream formulation or a powder formulation is applied on a surface, the present invention provides a novel composition and method, wherein a cream, applied on a surface, dries to a visible powder layer. After application on the surface, the cream dries to the visible powder layer preferably within about 10 seconds to 30 minutes, more preferably within about 20 seconds to 15 minutes and most preferably within about 30 seconds to 5 minutes. The visible powder layer acts as an active agent carrier, a moisture barrier, a protective film and a visual indicator of product site of application. For example, an appropriate amount of cream-active agent, applied to the buttocks of a baby, dries to a visible powder layer that provides an active agent delivery carrier, a moisture barrier, a protective film and a visual marker of the area of cream application. Moreover, as the visible powder layer is non-greasy and non-staining and is not displaced by movement, contact or abrasion, it can be covered with a diaper without concern that the composition will be rubbed off or absorbed into the diaper.

Also, in contrast to prior art methods, wherein an active agent is applied on a surface in either a cream formulation or in a powder formulation, the present invention provides a novel composition and method, wherein a cream-active agent is applied on a surface and dries to a visible powder layer. The active agent is chemically or physically incorporated into the cream-active vehicle in a soluble or as an insoluble suspension, or a combination of both, such that, as the cream dries to a visible powder layer, the active agent remains in stationary contact with the surface for a time sufficient to be effective. Active agents for use in the cream-active agent formulation include therapeutically active agents that are contained within the visible powder layer in a dry or wet state and are covered or surrounded by the visible powder layer. For use, an amount of a cream-active agent, containing an effective amount of the active agent, is applied on a defined small or large area of the skin, one or more times until a desired result is obtained. The visible powder layer is not displaced from the surface by movement, contact or abrasion.

In one embodiment, the present invention is useful for treatment of dermatological conditions, wherein a cream-pharmaceutical agent is applied on a defined area of skin and dries to a visible powder layer. The pharmaceutical active agent is either chemically or physically incorporated in the cream in a soluble or as an insoluble suspension, or a combination of both, such that, as the cream dries to a visible powder layer, the pharmaceutical active agent is maintained in stationary contact with the surface for a time sufficient to be effective. For use, an amount of a cream-pharmaceutical agent, containing an effective amount of the pharmaceutical agent, is applied on a defined area of skin one or more times until a desired result is obtained. Moreover, as the visible powder layer is non-greasy and non-staining and is not displaced by movement, contact or abrasion, it can be covered with clothing.

In an example to treat acne, a cream containing an effective amount of an agent for treating acne, such as the pharmaceutical agent salicylic acid, is applied on the area to be treated, such as the face, chest or back. The cream dries to a visible powder layer that is non-greasy, non-staining, abrasion resistant and that enables visual identification of the area of application. After the cream dries to a powder, the cream may be used inconspicuously during the day under clothing without rubbing off onto the clothing or staining the clothing. The cream may be used overnight on all areas, including the face, without rubbing off on bedclothes or pillows. Other types of acne treating agents are well-known in the art and are included in this invention. The cream/powder composition can be removed using a mild surfactant and water.

In an example to treat rectal itching, a cream containing an effective amount of an analgesic, such as Pramoxine HCL, is applied to the rectal area and dries to a visible powder layer that is non-greasy, non-staining, abrasion resistant and moisture absorbing. After the cream dries to a powder, the cream may be used inconspicuously during the day under clothing without rubbing off onto the clothing or staining the clothing and overnight without rubbing off on bedclothes. Other types of analgesic agents for treating hemorrhoids and rectal itching are well-known in the art and are included in this invention. The cream/powder composition can be removed using a mild surfactant and water. The cream-analgesic offers an advantage over currently marketed products such as ANUSOL® and PREPARATION H® that are petrolatum based ointments and, therefore are greasy, staining, non-moisture absorbing and do not offer a stationary localization on the skin of the active agent in suspension.

In an example to treat poison ivy or poison oak, a cream containing an effective amount of Hydrocortisone, is applied to the affected area and dries to a visible powder layer that is non-greasy, non-staining, non-scaly and that can easily be covered by clothing. After the cream dries to a powder, the cream may be used inconspicuously during the day under clothing without rubbing off onto the clothing or staining the clothing. The cream may be used overnight on all areas, including the face, without rubbing off on bedclothes or pillows. Other types of agents for treating poison ivy or poison oak are well-known in the art and are included in this invention. The cream-hydrocortisone/powder can be removed using a mild surfactant and water. The cream-hydrocortisone/powder offers an advantage over currently marketed hydrocortisone ointments products that are messy, staining, that cannot easily be covered by clothing, do not offer a stationary localization on the skin for the active agent in suspension.

In an example to treat allergic itchy skin conditions, a cream containing an effective amount of Diphenhydramine HCL, is applied to the affected area and dries to a visible powder layer that is non-greasy, non-staining, non-scaly and that can easily be covered by clothing. After the cream dries to a powder, the cream may be used inconspicuously during the day under clothing without rubbing off onto the clothing or staining the clothing. The cream may be used overnight on all areas, including the face, without rubbing off on bedclothes or pillows. Other types of agents for treating allergic itchy skin conditions are well-known in the art and are included in this invention. The cream-Diphenhydramine/powder offers an advantage over currently marketed products such as BENADRYL LOTION™ by providing long lasting skin protective effects and visual monitoring of site of application. The cream-Diphenhydramine/powder can be removed using a mild surfactant and water.

In an example to treat itchy skin conditions associated with fungal conditions such as Athlete's Foot, a cream containing an effective amount of miconazole nitrate, is applied to the affected area and dries to a visible powder layer that is non-greasy, non-staining, non-scaly and that can easily be covered by clothing. After the cream dries to a powder, the cream may be used inconspicuously during the day under clothing without rubbing off onto the clothing or staining the clothing and may be used overnight without rubbing off on bedclothes. Other types of fungal treating agents are well-known in the art and are included in this invention. The cream-anti-fungal/powder can be removed using a mild surfactant and water. The cream-antifungal/powder offers an advantage over currently marketed Miconazole powder products in that it is soothing, abrasive resistant, and long lasting.

In an example to treat chapped, dry skin conditions associated with diabetes, a cream containing an effective amount of urea, is applied to the affected area and dries to a visible powder layer that is non-greasy, non-staining, non-scaly and that can easily be covered by clothing. After the cream dries to a powder, the cream may be used inconspicuously during the day under clothing without rubbing off onto the clothing or staining the clothing. The cream may be used overnight on all areas, including the face, without rubbing off on bedclothes or pillows. Other types of agents for treating chapped, dry skin are well-known in the art and are included in this invention. The cream-urea/powder can be removed using a mild surfactant and water. The cream-urea/powder offers an advantage over currently marketed urea cream and ointment products in that it is soothing, abrasive resistant, moisture absorbing, and long lasting.

In another embodiment, the present invention is useful for the protection from dermatological conditions, wherein the cream without a pharmaceutical agent is applied on a defined area of skin and dries to a visible powder layer. For use, an amount of a cream is applied on a defined area of skin to protect against possible conditions, such as contact dermatitis, allergic reaction, bed sores from laying in one position for too long, irritated skin from urinary incontinence or diaper rash. Moreover, as the visible powder layer is non-greasy and non-staining and is not displaced by movement, contact or abrasion, it can be covered with clothing.

In an example to protect against diaper rash, an effective amount of the cream for protecting the skin from the irritants found in urine or fecal material is applied on the area to the diaper area. The cream dries to a visible powder layer that is non-greasy, non-staining, abrasion resistant and that enables visual identification of the area of application. After the cream dries to a powder, the cream may be used without rubbing off onto the diaper or being absorbed by the diaper. The cream/powder composition can be removed using a mild surfactant and water.

In an example to protect against bed sores, an effective amount of the cream for protecting the skin from bed sore ulcers is applied on the area to the diaper area. The cream dries to a visible powder layer that is non-greasy, non-staining, abrasion resistant and that enables visual identification of the area of application. After the cream dries to a powder, the powder will not rub off onto or be absorbed by the bedclothes. The cream/powder composition can be removed using a mild surfactant and water. Alternatively, the cream may have a vasodilator added as a therapeutic agent, to assist in the prevention of bed sore ulcers.

In another embodiment, the present invention is useful for the delivery of therapeutics through the skin for the treatment of conditions other than dermatological, wherein a cream-pharmaceutical agent is applied on a defined area of skin and dries to a visible powder layer. The pharmaceutical active agent is either chemically or physically incorporated in the cream in a soluble or as an insoluble suspension, or a combination of both, such that, as the cream dries to a visible powder layer, the pharmaceutical active agent is maintained in stationary contact with the surface for a time sufficient to be effective. For use, an amount of a cream-pharmaceutical agent, containing an effective amount of the pharmaceutical agent, is applied on a defined area of skin one or more times until a desired result is obtained. Moreover, as the visible powder layer is non-greasy and non-staining and is not displaced by movement, contact or abrasion, it can be covered with clothing.

In an example to soothe muscle aches, a cream containing an effective amount of an agent for treating muscle aches, such as the pharmaceutical agents of capsaicin, methyl salicylate, or methyl nicotinate, is applied on the area to be treated, such as the shoulders, chest or back. The cream dries to a visible powder layer that is non-greasy, non-staining, abrasion resistant and that enables visual identification of the area of application. After the cream dries to a powder, the cream may be used inconspicuously during the day under clothing without rubbing off onto the clothing or staining the clothing. The cream may also be used overnight without rubbing off on bedclothes or pillows. Other types of pain treating agents are well-known in the art and are included in this invention. The cream/powder composition can be removed using a mild surfactant and water.

In an example, to treat psoriatic skin conditions, a cream containing an effective amount of anthralin, is applied to the affected area and dries to a visible powder layer that is non-greasy, non-staining, non-scaly and that can easily be covered by clothing. The cream-anthralin offers an advantage over currently marketed products such as DITHRANOL™ by providing long lasting skin protective effects and visual monitoring of site of application. This is especially important as anthralin may irritate the skin, and visual monitoring will help with removal of the therapeutic when treatment is complete. The cream-base of the cream-anthralin composition also slows down the delivery of the sometimes irritating therapeutic. The cream-anthralin can be removed using a dermatological lotion like Cetaphyl Lotion™ or mild surfactant and water on the alkaline side of pH.

In an example, to treat hyper-pigmentation conditions of the skin associated dark spots and age spots related to extensive exposure to sunlight, a cream containing an effective amount of hydroquinone, is applied to the affected area and dries to a visible powder layer that is non-greasy, non-staining, non-scaly and that can easily be covered by clothing. The cream-hydroquinone can be removed using a mild surfactant and water. The cream-hydroquinone offers an advantage over currently marketed skin lightning products in that it is soothing, abrasive resistant, long lasting and visible monitors site of application.

The cream component of the present invention comprises one or more non-swelling clays, swelling clays, natural gums, non-ionic surfactants, hydrophilic and/or hydrophobic solvents, each of which is toxicologically, pharmaceutically and generally regarded as safe (GRAS) according to U.S. Federal Food and Drug Regulation Standards. If an active agent is part of the composition, the active agent is a pharmaceutical agent.

The compounds listed below are those of standard use in the art. The examples provided do not limit the types of compounds to be used in the invention, but are merely illustrative. Other compounds of standard use and non-standard use in the art may also be used.

Non-swelling clays include, but are not limited to, kaolin. Swelling clays include, but are not limited to, montmorillonite clays. Montmorillonite clays include, but are not limited to, magnesium aluminum silicate, bentonite, hectorite, or surface reactive montmorillonite clays including, but not limited to, stearalkonium hectorites. Natural gums include, but are not limited to, xanthan gum, guar gum, gum arabic, carrageenan and alginates. Alginates include, but are not limited to, alginic acid and sodium alginate. Synthetic gums include but are not limited to acrylic polymers. Non-ionic surfactants include, but are not limited to, stearates, palmitostearates, glycerides, sorbitan esters, polyoxyethylene ethers, polyoxyethylene glycol esters, C₂₀-C₂₄ glyceryls and emulsifying waxes. Stearates include those with melting ranges between about 33.0° and 97.5° C. such as, but not limited to, polyethylene-6 stearate, polyethylene-35 stearate, glyceryl stearate, glycol stearate and polyglycol stearate (average MW ≧300). Palmitosterates include those with melting ranges between about 26.5° and 62.0° C. such as, but not limited to, glyceryl palmitostearates, polyglycol (average MW ≧300), palmitostearates and propylene glycol palmitostearates. Glycerides include those with melting ranges between about 29.0° to 70.0° C. such as, but not limited to, saturated polyclycolised glycerides (GELUCIRE, Gattefosse Corp, Westwood, N.J.) with melting ranges between about and 31.0° to 45.0° C., C₁₂-C₁₈ glycerides, hemisynthetic glycerides and caproyl/capric glycerides. Sorbitan esters include those with melting points ≧25.5° C. such as, but not limited to, sorbitan palmitate, sorbitan stearate and sorbitan tristearate. Polyoxyethylene ethers include those with melting points ≧26.5° C. such as, but are not limited to, laureth-12, ceteth-2, steareth-2, oleth-20, ceteareth-4, trideceth-12, and PEG-40 hydrogenated castor oil. Polyoxyethylene glycol esters include those with melting points ≧26.5° C. such as, but not limited to, PEG-8 stearate, PEG-100 stearate, PEG-8 distearate, and PEG-150 distearate. C₂₀-C₂₄ glyceryls include those with melting points ≧26.5° C. such as, but not limited to, glyceryl behenate and glyceryl tribehenate. Emulsifying waxes include those with melting points ≧26.5° C. such as, but not limited to, emulsifying wax NF, cetearyl alcohol and ceteareth-20, stearyl alcohol and ceteareth-20, cetearyl alcohol and ceteth-20. Hydrophilic solvents include, but are not limited to, water, glycerin, propylene glycol and polyethylene glycol (average MW<600 Da). Natural or Synthetic film formers include but are not limited to chitosan, albumin, hyaluronic acid and poly vinyl pyrilidone (PVP). Hydrophobic solvents include, but not limited to hydrocarbons such as mineral oil, esters such as isopropyl myristate, and silicone ingredients such as dimethicone.

By percent weight, the cream-active of the present invention comprises non-swelling clay preferably at about 5 to 60%, more preferably at about 10 to 50% and most preferably at about 15 to 40%; swelling clay preferably at about 0.2 to 20%, more preferably at about 0.5 to 10% and most preferably at about 1 to 5%; non-ionic surfactant preferably at about 0.5 to 30%, more preferably at about 1 to 20% and most preferably at about 2 to 15%; natural or synthetic gum preferably at about 0.01 to 5%, more preferably at about 0.02 to 2% and most preferably at about 0.05 to 1%; a natural or synthetic film former at about 0.01 to 1%, more preferably at about 0.02 to 0.5%, and most preferably at about 0.1 to 0.25%; a hydrophilic and/or hydrophobic solvent to 0.1 to 20%, and a therapeutically-active agent at about 0.001 to 50%. The viscosity of the cream is preferably between about 10,000 and 15,000,000 centipoise, more preferably between about 50,000 and 12,500,000 centipoise and most preferably between about 100,000 and 10,000,000 centipoise.

Optional ingredients for use in the cream of the present invention include, but are not limited to, water insoluble powders, preservatives and humectants. Water insoluble powders include, but are not limited to, talc, starch, starch derivatives, polymers, zinc oxide, titanium dioxide, silicon dioxide, metal stearates, mica and combinations thereof. Metal stearates include, but are not limited to, magnesium stearate and zinc stearate. Preservatives include, but are not limited to, benzyl alcohol, boric acid, chloroxylenol, dehydroacetic acid, orthophenylphenol, quatemium-15, ethanol, diazolidinyl urea, imidazolidinyl urea, hydantion, benzethonium chloride, potassium sorbate, sodium benzoate, phenoxyethanol, phenylethyl alcohol, methyl paraben, ethyl paraben, propyl paraben, butyl paraben, sorbic acid, glutaraldehyde and combinations thereof. Humectants include, but are not limited to, glycerin, hexylene glycol, propylene glycol, sorbitol, fructose, glucose, lactose, polyammio sugar condensate, hydrogenated starch hydrolysate, acetamide DEA, glycereth-7, honey, methyl gluceth-10, PCA, sodium lactate, saccharide hydrolysate, xylitol, botanical extracts and combinations thereof.

Other optional ingredients include, but are not limited to, pigments, emollients, acidifying agents, propellants, alcohols, alkalinizing agents, antifoaming agents, antioxidants, buffering agents, suspending agents, chelating agents, coating agents, complexing agents, coloring agents, desiccants, flavors, perfumes, plasticizers, solvents, sorbents, penetration enhancers, stiffening agents, tonicity agents, thickening agents, viscosity increasing agents, sweetening agents, oils, solid carriers, water repelling agents, wetting agents, solubilizing agents, natural polymers, synthetic polymers and combinations thereof. Natural polymers and synthetic polymers include, but are not limited to, acrylic copolymers, methacrylates, hydroxypropyl cellulose, hydroxyethylcellulose and hydroxypropylmethyl cellulose.

Active agents for use in the cream-active agent include, but are not limited to, natural agents, synthetic agents, chemical agents, pharmaceutical agents and combinations thereof that are completely soluble, partially soluble or insoluble in aqueous solution. Pharmaceutical agents include, but are not limited to, analgesics, anesthetics, antibiotics, anti-infectives, antimicrobials, anti-inflammatories, anti-neoplastics, antirheumatics, antivirals, antifungals, anticomediforms (acne), antihistamines, antialopecias, antipruritics, antipsoriatics antiseborrheics, anti-herpetiformes, enzymes, keratolitics, steroids, local anesthetics, antiseptics disinfectants, pigmentors depigmentors, hair growth stimulants, vasodilators, rubefacients, protectants and sunscreens. Pharmaceutical agents include, but are not limited to itraconazole, miconazole, fluconazole, ketoconazole, tetracaine, lidocaine, mepivicaine, phenol, pramoxine, prilocaine, procaine, benzoyl peroxide, resorcinol, retinoic acid, histamine, poison ivy extract, beclomethasone, dexamethasone, minoxidil, neomlycin, tolnaftate, undecylenic acid, salicylic acid, nimesulide, camphor, menthol, anthralin, pyrithione, selenium sulfide, alcohols, chlorbexidine, bismuth subgallate, sulfur, triclosan, hydrogen peroxide, chlorxylenol, hexachlorophene, chlorhexidine and chlorhexidine salts, benzethonium chloride, silver nitrate, boric acid, acyclovir, hydroquinone, allantoin, calamine, titanium dioxide, zinc, oxide, dimethicone, PABA, oxybenzone dithranol, salicylates, griseofulvin, vitamins A, vitamin D, vitamin E, proteolytic enzymes, mucolytic enzymes, corticosteroids, hydrocortisone sunscreens, protectants, aloe, capsaicin, chamomile, panthenol, fluticazone diproprionate, coal tar, alpha hydroxy acids, beta hydroxy acids, ammonium lactate, calcipotriene, 5-fluorouracil.

Optional color indicators for use in the cream-active agent include any color indicator substance, not limited to any pigment, pH indicator, or gram negative and gram positive color indicators such as crystal violet, Iodine, sefranin and combinations thereof.

In an example, a cream-active agent is applied on a defined area of skin and, dries to a visible powder from which the active agent is delivered onto the epidermal layer of the skin. In another example, a cream-active agent is applied on a defined area of skin and dries to a visible powder layer from which the active agent is delivered through the epidermal layer into the dermal layer of the skin.

The cream-active agent is useful for the prevention and treatment of diseases and disorders of the skin of an animal, including a human, and, more importantly, for the prevention and treatment of diseases and disorders affecting all parts of an animal, including a human. Such diseases and disorders include, but are not limited to, muscle pain, joint pain, headache, migraine, bacterial infection, fungal infection, tinea capitis, tinea corporis, tinea cruris, tinea versicolor, viral infection, microbial infections, allergic reaction, actinic cheilitis, herpes, wounds, burns, warts, diaper rash, psoriasis, acne, seborrheic dermatitis, contact dermatitis, allergic dermatitis, hemorrhoids, wrinkles, insect bites, itching, sunburn, razor burn, eczema, psoriasis, chicken pox, rashes, blemishes and other diseases and disorders.

The cream-active agent used, the number of applications and the schedule of application will depend on the disease or disorder being prevented or treated, the severity of the disease or disorder, the location of the disease or disorder and other clinical factors such as the size and physical condition of the recipient and can be determined by the medical practitioner using standard clinical techniques and without undue experimentation. The cream-active agent is applied to a defined surface area preferably 1 to 10 times per 24 hours, more preferably 3 to 6 times per 24 hours and most preferably 2 to 5 times per 24 hours. The amount of cream applied is preferably between about 1 and 100 mg/cm², more preferably between about 10 and 90 mg/cm² and most preferably between about 20 and 70 mg/cm². The amount of active agent in the cream depends on the condition to be treated, individually characteristics of the patient and the strength of the active agent. Guidelines for dosage are those that are standard in the art. Generally, the amount of active agent in the cream is preferably from about 0.001 to 50%, more preferably from about 0.01 to 30%, and most preferably from about 0.1 to 10%. The amount of active agent per application is preferably between about 0.05 and 500 mg, more preferably between about 0.1 and 300 mg and most preferably between about 0.5 and 100 mg. However, variations may be required based on individual factors.

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### EXAMPLE 1

Preparation of a Cream-Active I—Product for Athlete's Foot

In an embodiment of the present invention, a cream-active agent containing the pharmaceutical agent miconazole nitrate USP was prepared as follows. NF is the National Formulary.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s. to 100% |
| PEG-6 stearate & PEG-32 stearate | 5.0 |
| Ethoxydiglycol | 4.0 |
| Sorbic Acid NF | 0.1 |
| PVP | 0.02 |

Part B

| | |
|---|---|
| Purified water USP | 1.0 |
| Imidurea NF | 0.3 |

Part C

| | |
|---|---|
| Miconazole nitrate USP | 2.0 |

1. Xanthum gum NF (KETROL F, Monsanto, Chicago, Ill.) and the magnesium aluminum silicate NF (VEEGUM ULTRA, RT Vanderbilt, Norwalk, Conn.) were dry blended in a suitably sized blender.

2. Purified water USP was charged into a suitably sized jacketed stainless steel kettle equipped with a Lightnin type mixer (VWR Scientific, Pittsburgh, Pa.) and a double-motion impeller and was heated to between 73° and 77° C. During heating, the purified water USP was mixed vigorously with the Lightnin type mixer.

3. The step 1 mixture was dispersed into the purified water USP by sprinkling it into the vortex of the mixing water and mixing was continued for 60 to 75 minutes, followed by mixing with a double motion impeller.

4. While maintaining temperature and mixing conditions, the following ingredients were added in order: kaolin USP (VANCLAY, RT Vanderbilt, Norwalk, Conn.), talc USP (MIRAGE 500, Ultra, Red Bank, N.J.), PEG-6 stearate and PEG-32 Stearate (TEFOSSE 1500, Gattefosse Westwood, N.J.), glycerin USP (Jeen International, Little Falls, N.J.), sorbic acid NF (Eastman, Kingsport Tenn.) and potassium sorbate NV (Jeep International, Little Falls, N.J.).

5. After thorough mixing of all ingredients, the mixture (step 4) was cooled to 50° C. with constant mixing.

6. The imidurea NF (GERMALL 115, ISP, Wayne, N.J.) and purified water USP of Part B were mixed together and were added to the mixture of step 5 with constant mixing.

7. The miconazole nitrate USP (Zetapharm, New York, N.Y.) of Part C was slowly mixed into the step 6 mixture, and mixing was continued until a uniform pasty cream was obtained.

8. The mixture was cooled to below 40° C. with continued mixing until a uniform pasty cream is obtained.

The cream is packaged into individual aluminum tubes of between 4 and 60 mgs using methods known to those skilled in the art. All steps in the preparation and packaging are performed under aseptic conditions.

The viscosity of the cream-miconazole nitrate was 1,200,000 cps as tested by Brookfield viscometer Model LVT, Spindle 7, 0.05 RPM.

The cream-active agent is packaged in aluminum tubes of between 4 and 60 grams using methods known to those skilled in the art. All steps in the preparation and packaging are performed under aseptic conditions. The product delivers miconazole nitrate onto the skin for extended periods of time.

### EXAMPLE 2

Stability of Cream-Active Agent I

Example 1 cream-miconazole nitrate was packaged in 15 g aluminum tubes and stored at 40° C. for 3 months. The stability of Example 1 cream-miconazole nitrate was assayed a 0 (initial) 1, 2 and 3 month intervals using the stability assay for miconazole nitrate cream described in the United States Phairmacopeia XXII at page 898. The results are shown in Table 1.

TABLE 1
Stability of Example 1 cream-miconazole nitrate

| | | 1- | 2- | 3- |
|---|---|---|---|---|
| TEST | INITIAL | MONTH | MONTHS | MONTHS |
| ASSAY(% of Initial) | 100 | 99 | 100 | 105 |
| pH | 4.8 | 4.9 | 5.0 | 5.0 |
| Appearance | | *UC | UC | UC |

*unchanged from initial appearance of a light-brown pasty cream.

These data demonstrate that the miconazole nitrate in the cream-miconazole nitrate of Example 2 remained stable over 3 months at 40° C. and that the pH of the cream-miconazole nitrate remained stable over the 3 month period. As defined in Remington's Pharmaceutical Sciences (Mack Publishing Co., Easton, Pa. 1990, p. 1504), a product needs to retain retain ≧90% of its labeled value as the minimal acceptable level of stability. A product that retains ≧90% of its labeled value over 3 months at accelerated conditions (40° C.) enables a manufacturer to claim 2 years shelf life at room temperature (25° C.) (FDA Guidance for Industry: Stability testing of Drug Substances and Drug Products).

### EXAMPLE 3

Preparation of Cream-Active Agent II—Product for Rectal Itch

In an embodiment of the present invention, a cream-active agent containing pharmaceutical agent pramoxine hydrochloride USP was prepared as follows.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s. to 100% |
| PEG-6 stearate & PEG-32 stearate | 5.0 |
| Ethoxydiglycol | 4.0 |
| Methylparaben NF | 0.3 |
| Propylparaben NF | 0.15 |
| Sorbic Acid NF | 0.1 |
| PVP | 0.02 |

Part B

| | |
|---|---|
| Purified Water USP | 1.0 |
| Imidurea NF | 0.15 |

Part C

| | |
|---|---|
| Pramoxine hydrochloride USP | 1.0 |

Part A and Part B ingredients were mixed together as in steps 1-6 of Example 1. The pramoxine hydrochloride USP (Abbot, North Chicago, Ill.) of Part C was slowly mixed into the step 6 mixtures as in step 7 of Example 2 and mixing was continued until a uniform pasty cream was obtained. The viscosity of the cream-pramoxine hydrochloride was 640,000 cps as tested by Brookfield viscorneter, Model LVT, Spindle 7, 0.05 RPM.

The cream is packaged into tubes or jars by methods known to those skilled in the art. All steps in the preparation and packaging are performed under aseptic conditions. The product delivers Pramoxine HCL into the skin for extended periods of time.

### EXAMPLE 4

Stability of Cream-Active Agent II

Example 3 cream-pramoxine hydrochloride was packaged in 15 g aluminum tubes and stored at 40° C. during the 3 months. The stability of Example 3 cream-pramoxine hydrochloride was assayed a 0 (initial) 1, 2 and 3 month intervals using the stability assay for pramoxine hydrochloride cream described in the United States Pharmacopoeia XXII at page 1122. The results are shown in Table 2.

TABLE 2
Stability of Example 4 cream-pramoxine hydrochloride

| | | 1- | 2- | 3- |
|---|---|---|---|---|
| TEST | INITIAL | MONTH | MONTHs | MONTHS |
| ASSAY (% of initial) | 1.12% | 1.15% | 1.01% | 1.00% |
| pH | 5.1 | 4.6 | 4.6 | 4.8 |
| Appearance | | *UC | UC | UC |

*unchanged from initial appearance of a light-brown pasty cream.

These data demonstrate that the pramoxine hydrochloride in Example 4 cream-pramoxine hydrochloride remained stable over 3 months at 40° C. and that the pH of the cream-pramoxine hydrochloride remained stable over the 3 month period. The pH of the cream-miconazole nitrate remained stable over the 3 month period.

### EXAMPLE 5

Preparation of Cream-Active Agent III—Product for Allergic Itch Skin Conditions

In an embodiment of the present invention, a cream-active agent containing pharmaceutical agent Diphenhydramine Hydrochloride was prepared as follows.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s. to 100% |
| Polowax NF | 5.0 |
| Glycerin USP | 4.0 |
| Methylparaben NF | 0.3 |
| Propylparaben NF | 0.15 |
| Sorbic Acid NF | 0.1 |
| Chitosan | 0.2 |

Part B

| | |
|---|---|
| Purified water USP | 1.0 |
| Imidurea NF | 0.15 |

Part C

| | |
|---|---|
| Diphenhydramione hydrochloride USP | 1.0 |

Part A and Part B ingredients were mixed together as in steps 1-6 of Example 1. The Diphenhydramine hydrochloride USP (Sigma) of Part C was slowly mixed into the step 6 mixtures as in step 7 of Example 2 and mixing was continued until a uniform pasty cream was obtained. The viscosity of the cream-Diophenhydramine hydrochloride was 840,000 cps as tested by Brookfield viscometer, Model LVT, Spindle 7, 0.05 RPM.

The cream is packaged into tubes or jars by methods known to those skilled in the art. All steps in the preparation and packaging are performed under aseptic conditions.

### EXAMPLE 6

Preparation of Cream-Active Agent IV—Product for Diabetic Foot

In an embodiment of the present invention, a cream-active agent containing pharmaceutical agent Urea USP was prepared as follows.

| | |
|---|---|
| INGREDIENT | % w/w |

Part A

| | |
|---|---|
| Xanthum gum NF | 0.2 |
| Magnesium aluminum silicate NF | 3.5 |
| Kaolin USP | 21.0 |
| Talc USP | 6.0 |
| Purified water USP | q.s to 100% |
| PEG-6 Stearate & PEG-32 Stearate | 5.0 |
| Pentalene Glycol | 4.0 |
| Methylparaben NF | 0.3 |
| Propylparaben NF | 0.15 |
| Isopropyl Myristate NF | 1.0 |
| Hyaluaronic acid | 0.02 |

Part B

| | |
|---|---|
| Purified water USP | 1.0 |
| Imidurea NF | 0.15 |

Part C

| | |
|---|---|
| Urea USP | 20.0 |

Part A and Part B ingredients were mixed together as in steps 1-6 of Example 1. The Urea USP (EM Industries) of Part C was slowly mixed into the step 6 mixtures as in step 7 of Example 1 and mixing was continued until a uniform pasty cream was obtained. The viscosity of the cream-urea was 700,000 cps as tested by Brookfield viscometer, Model LVT, Spindle 7, 0.05 RPM.

The cream is packaged into tubes or jars by methods known to those skilled in the art. All steps in the preparation and packaging are performed under aseptic conditions. The product is tested on 100 subjects under a Repeat Insult Patch Test (RIPT) and demonstrate no significant clinical indication to cause irritation.

### EXAMPLE 7

Preparation of Cream-Active Agent V—Skin Protection Product Against Contact Dermatitis

In an embodiment of the present invention, a cream-active agent containing pharmaceutical agent Benzothonium Chloride was prepared as follows.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s to 100% |
| PEG-6 stearate & PEG-32 stearate | 5.0 |
| Glycerin USP | 4.0 |
| Methylparaben NF | 0.3 |
| Propylparaben NF | 0.15 |
| Sorbic Acid NF | 0.1 |
| Albumin | 0.02 |

Part B

| | |
|---|---|
| Purified water USP | 1.0 |
| Imidurea NF | 0.15 |

Part C

| | |
|---|---|
| Benzothonium Chloride | 0.2 |

Part A and Part B ingredients were mixed together as in steps 1-6 of Example 1. The Benzothonium Chloirde of Part C was slowly mixed into the step 6 mixtures as in step 7 of Example 1 and mixing was continued until a uniform pasty cream was obtained. The viscosity of the cream-Benzothonium was 700,000 cps as tested by field viscometer, Model LVT, Spindle 7, 0.05 RPM.

The cream is packaged into tubes or jars by methods known to those skilled in the art. All steps in the preparation and packaging are performed under aseptic conditions. There is no incompatibility between the base and the cationic active agent.

### EXAMPLE 8

Preparation of Cream-Active Agent VI—Anti-Microbial Product for under Latex Glove Use

In an embodiment of the present invention, a cream-active agent containing pharmaceutical agent Triclosan was prepared as follows.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s. to 100% |
| PEG-6 stearate & PEG-32 stearate | 5.0 |
| Glycerin USP | 4.0 |
| Methylparaben NF | 0.3 |
| Propylparaben NF | 0.15 |
| Sorbic Acid NF | 0.1 |
| PVP | 0.2 |

Part B

| | |
|---|---|
| Purified water USP | 1.0 |
| Imidurea NF | 0.15 |

Part C

| | |
|---|---|
| Triclosan USP | 1.0 |

Part A and Part B ingredients were mixed together as in steps 1-6 of Example 1. The Triclosan USP of Part C was slowly mixed into the step 6 mixtures as in step 7 of Example 1 and mixing was continued until a uniform pasty cream was obtained. The viscosity of the cream-triclosan was 1,200,000 cps as tested by Brookfield viscometer, Model LVT, Spindle 7, 0.05 RPM.

The cream is packaged into tubes or jars by methods known to those skilled in the art. All steps in the preparation and packaging are performed under aseptic conditions.

### EXAMPLE 9

Preparation of Cream-Active Agent VII—Acne Scars—Physician TCA Skin Peel Mask

In an embodiment of the present invention, a cream-active agent containing pharmaceutical agent Trichloracetic acid was prepared as follows.

| | |
|---|---|
| INGREDIENT | % w/w |

Part A

| | |
|---|---|
| Xanthum gum NF | 0.2 |
| Magnesium aluminum silicate NF | 3.0 |
| Kaolin USP | 29.0 |
| Talc USP | 6.0 |
| Purified water USP | q.s. to 100% |
| PEG-6 stearate & PEG-32 stearate | 5.0 |
| Ethoxidiglycol | 7.0 |
| Methylparaben NF | 0.3 |
| Propylparaben NF | 0.15 |
| Chitosan | 0.1 |
| Bisabolol | 0.2 |

Part B

| | |
|---|---|
| Purified water USP | 1.0 |
| Imidurea NF | 0.15 |

Part C

| | |
|---|---|
| Trichloracetic acid USP | 1.0 |

Part A and Part B ingredients were mixed together as in steps 1-6 of Example 1. The Triclosan USP of Part C was slowly mixed into the step 6 mixtures as in step 7 of Example 1 and mixing was continued until a uniform pasty cream was obtained. The viscosity of the cream-triclosan was 900,000 cps as tested by Brookfield viscometer, Model LVT, Spindle 7, 0.05 RPM. The cream is packaged into tubes or jars by methods known to those skilled in the art. All steps in the preparation and packaging are performed under aseptic conditions. The active agent is delivered into the skin. Product is used for 10-20 minutes and removed by rinsing with water and mild surfactant.

### EXAMPLE 10

Preparation of Cream-Active Agent VIII—Psoriasis—Anthralin Product

In an embodiment of the present invention, a cream-active agent containing pharmaceutical agent Anthralin was prepared as follows.

| | |
|---|---|
| INGREDIENT | % w/w |

Part A

| | |
|---|---|
| Xanthum gum NF | 0.3 |
| Magnesium aluminum silicate NF | 3.0 |
| Kaolin USP | 28.0 |
| Talc USP | 6.0 |
| Purified water USP | q.s. to 100% |
| PEG-6 stearate & PEG-32 stearate | 5.0 |
| Propylene Glycol USP | 4.0 |
| Methylparaben NF | 0.3 |
| Propylparaben NF | 0.15 |
| Sorbic Acid NF | 0.5 |
| PVP | 0.2 |

Part B

| | |
|---|---|
| Purified water USP | 1.0 |
| Imidurea NF | 0.15 |

Part C

| | |
|---|---|
| Anthralin USP | 1.0 |

Part A and Part B ingredients were mixed together as in steps 1-6 of Example 1. The Anthralin USP of Part C was slowly mixed into the step 6 mixtures as in step 7 of Example 1 and mixing was continued until a uniform pasty cream was obtained. The viscosity of the cream-anthralin was 900,000 cps as tested by Brookfield viscometer, Model LVT, Spindle 7, 0.05 RPM. The cream is packaged into tubes or jars by methods known to those skilled in the art. All steps in the preparation and packaging are performed under aseptic conditions. The active agent is delivered onto the skin. Product is used for 10-20 minutes and removed by rinsing with water and mild alkaline pH surfactant.

### EXAMPLE 11

Preparation of Cream-Active Agent IX—Poison Ivy—Hydrocortisone Product

In an embodiment of the present invention, a cream-active agent containing pharmaceutical agent Hydrocortisone was prepared as follows.

| | |
|---|---|
| Ingredient | % w/w |

Part A

| | |
|---|---|
| Xanthum gum NF | 0.4 |
| Magnesium aluminum silicate NF | 2.5 |
| Kaolin USP | 27.0 |
| Talc USP | 4.0 |
| Purified water USP | q.s. to 100% |
| PEG-6 stearate & PEG-32 stearate | 4.0 |
| Ethoxidiglycol | 4.0 |
| Methylparaben NF | 0.3 |
| Propylparaben NF | 0.15 |
| Sorbic Acid NF | 0.5 |
| PVP | 0.2 |

Part B

| | |
|---|---|
| Purified water USP | 1.0 |
| Imidurea NF | 0.15 |

Part C

| | |
|---|---|
| Hydrocortisone USP | 1.0 |

Part A and Part B ingredients were mixed together as in steps 1-6 of Example 1. The Hydrocortisone USP of Part C was slowly mixed into the step 6 mixtures as in step 7 of Example 1 and mixing was continued until a uniform pasty cream was obtained. The viscosity of the cream-hydrocortisone was 1,100,000 cps as tested by Brookfield viscometer, Model LVT, Spindle 7, 0.05 RPM. The cream is packaged into tubes or jars by methods known to those skilled in the art. All steps in the preparation and packaging are performed under aseptic conditions. The active agent is delivered onto the skin. Product is used for long periods of time for extended release of hydrocortisone onto skin.

### EXAMPLE 12

Preparation of Cream-Active Agent X—Skin Lightning—Hydroquinoone Product

In an embodiment of the present invention, a cream-active agent containing pharmaceutical agent Hydroquinone was prepared as follows.

| | |
|---|---|
| Ingredient | % w/w |

Part A

| | |
|---|---|
| Xanthum gum NF | 0.4 |
| Magnesium aluminum silicate NF | 2.5 |
| Kaolin USP | 26.0 |
| Talc USP | 4.5 |
| Purified water USP | q.s. to 100% |
| PEG-6 stearate & PEG-32 stearate | 4.0 |
| 1,3 Butylene Glycol | 3.0 |
| Methylparaben NF | 0.3 |
| Propylparaben NF | 0.15 |
| Sodium Bisulfite USP | 0.6 |
| PVP | 0.2 |

Part B

| | |
|---|---|
| Purified water USP | 1.0 |
| Imidurea NF | 0.15 |

Part C

| | |
|---|---|
| Hydroquinone USP | 2.0 |

Part A and Part B ingredients were mixed together as in steps 1-6 of Example 1. The Hydroquinone USP of Part C was slowly mixed into the step 6 mixtures as in step 7 of Example 1 and mixing was continued until a uniform pasty cream was obtained. The viscosity of the cream-hydroquinone was 1,000,000 cps as tested by Brookfield viscometer, Model LVT, Spindle 7, 0.05 RPM. The cream is packaged into tubes or jars by methods known to those skilled in the art. All steps in the preparation and packaging are performed under aseptic conditions. The active agent is delivered onto the skin. Product is used for long periods of time for extended release of hydroquinone onto skin.

### EXAMPLE 13

Preparation of Cream-Active Agent XI—Microcirculation Product for Debicutis Ulcers

In an embodiment of the present invention, a cream-active agent containing pharmaceutical agent Methyl Nicotinate was prepared as follows.

| | |
|---|---|
| Ingredient | % w/w |

Part A

| | |
|---|---|
| Alginate gum | 0.5 |
| Magnesium aluminum silicate NF | 2.5 |
| Kaolin USP | 26.0 |
| Talc USP | 4.5 |
| Purified water USP | q.s. to 100% |
| PEG-6 stearate & PEG-32 stearate | 4.0 |
| 1,3 Butylene Glycol | 3.0 |
| Methylparaben NF | 0.3 |
| Propylparaben NF | 0.15 |
| Sorbic Acid NF | 0.5 |
| PVP | 0.2 |

Part B

| | |
|---|---|
| Purified water USP | 1.0 |
| Imidurea NF | 0.15 |

Part C

| | |
|---|---|
| Methyl Nicotinate USP | 0.2 |

Part A and Part B ingredients were mixed together as in steps 1-6 of Example 1. The Methyl Nicotinate USP of Part C was slowly mixed into the step 6 mixtures as in step 7 of Example 1 and mixing was continued until a uniform pasty cream was obtained. The viscosity of the cream-Nicotinate was 700,000 cps as tested by Brookfield viscometer, Model LVT, Spindle 7, 0.05 RPM. The cream is packaged into tubes or jars by methods known to those skilled in the art. All steps in the preparation and packaging are performed under aseptic conditions. The active agent is delivered onto the skin. Product is used for long periods of time for extended release of methylnicotinate onto skin.

We claim:

1 A composition, comprising:
a cream comprising 5 to 60% of a non-swelling clay, a swelling clay, a natural or synthetic gum, and a natural or synthetic film former,
wherein the cream dries to a visible, abrasion-resistant powder when applied on the skin of an animal.

2. The composition of claim 1 further comprising a non-ionic, anionic or cationic surfactant, a hydrophilic solvent, a hydrophobic solvent, or combinations thereof.

3. The composition of claim 1 further comprising a therapeutically active agent such that an amount of the therapeutically active agent effective to treat or prevent the disorder is delivered to the animal having the disorder.

4. A composition, comprising:
a cream comprising 5 to 60% of a non-swelling clay, a swelling clay, a natural or synthetic gum, a natural or synthetic film former and a therapeutically active agent such that an amount of the therapeutically active agent effective to treat or prevent the disorder is delivered to the animal having the disorder.

5. A method of preventing a disorder, comprising:
applying to the skin of an animal in a location susceptible to the disorder, a cream comprising 5 to 60% of a non-swelling clay, a swelling clay, a natural or synthetic gum, and a natural or synthetic film former; and
allowing the cream to dry to a visible, abrasion-resistant powder on the skin,
wherein the visible, abrasion resistant powder protects the skin from the disorder.

6. The method of claim 5 wherein the cream further optionally comprises a non-ionic, anionic or cationic surfactant, a hydrophilic solvent, a hydrophobic solvent, or combinations thereof.

7. The method of claim 5 where in the disorder is chosen from the group comprising contact dermatitis, diaper rash, incidents of dibicutis ulcers and poison oak or ivy.

8. The method of claim 5 wherein the cream further comprises a therapeutically active agent such that an amount of the therapeutically active agent effective to treat or prevent the disorder is delivered to the animal having the disorder.

9. The method of claim 8 wherein the disorder is chosen from the group comprising muscle pain, joint pain, headache, migraine, bacterial infection, fungal infection, tinea capitis, tinea corporis, tinea cruris, tinea versicolor, viral infection, microbial infections, allergic reaction, actinic cheilitis, herpes, wounds, burns, warts, diaper rash, psoriasis, acne, seborrheic dermatitis, contact dermatitis, allergic dermatitis, hemorrhoids, wrinkles, insect bites, itching, sunburn, razor burn, eczema, psoriasis, chicken pox, rashes, blemishes.

10. The method of claim 8 wherein the disorder is inflammation.

11. The method of claim 10 wherein the therapeutic agent is an anti-inflammatory.

12. The method of claim 8 wherein the disorder is fungal itch.

13. The method of claim 12 wherein the therapeutic agent is miconazole nitrate.

14. The method of claim 8 wherein the disorder is chapped, dry skin associated with diabetes.

15. The method of claim 14 wherein the therapeutic agent is urea.

16. The method of claim 8 wherein the disorder is allergic itchy skin conditions.

17. The method of claim 16 wherein the therapeutic agent is diphenhydramine HCL.

18. The method of claim 8 wherein the disorder is muscle ache.

19. The method of claim 12 wherein the therapeutic agent is chosen from the group comprising capsaicin, methyl salicylate, or methyl nicotinate.

20. The method of claim 8 further comprising the step of monitoring the location and duration of application of the therapeutic agent by observing the visible, abrasive-resistant powder.
